(19) United States
(12) Patent Application Publication
(10) Pub. No.: US 2002/0183596 A1
(43) Pub. Date: Dec. 5, 2002

(54) Candy coated tongue depressor

(76) Inventor: Basil Bruno, Mahwah, NJ (US)

Correspondence Address: ANTHONY D. CIPOLLONE, ESQ., One Essex Street, Hackensack, NJ, 07601, US

(21) Appl. No.: 09/872,909

(22) Filed: Jun. 4, 2001

(51) Int. Cl. A61B 1/32

(52) U.S. Cl. 600/240

(57) ABSTRACT

A candy coated tongue depressor manufactured from an edible material. The edible material is selected from a group consisting of candy, chocolate, fruit, fruit composite, vegetable, vegetable composite, flour, flour composite, ice, flavored ice, meat, meat composite, poultry, poultry composite, fish and fish composite. The tongue depressor, as an alternative, is manufactured from a non-edible material. The non-edible material is selected from a group consisting of wood, wood composite, plastic, plastic composite, rubber, rubber composite, metal, and metal alloy. The candy coated tongue depressor 10 further has a candy coating securely affixed to the outer surface of the tongue depressor rear. The candy is hard candy or chocolate.

### BACKGROUND OF INVENTION

1. Field of the Invention

The present invention relates to tongue depressors. More particularly, the present invention relates to coated tongue depressors.

2. Description of the Prior Art

During the physical examination of a child it is necessary to examine the mouth and throat. Traditionally, a tongue depressor is used to hold the tongue in a position so the doctor can see. Often the patient is not very cooperative. As an encouragement for the patient to cooperate the patient may be enticed with a reward. A typical reward might be candy or a lollipop. In the prior art various tongue depressors are known which have candy molded onto the body member or a candy member such as a lollipop is attached to the tongue depressor. This approach results in a specialized device which is expensive to produce and prevents manufacture in the standard manufacturing production lines. What is needed is a device, which uses as a base design, the standard tongue depressor and yet tastes like a candy.

Numerous innovations for a candy coated tongue depressor have been provided in the prior art that are described as follows. Even though these innovations may be suitable for the specific individual purposes to which they address, they differ from the present invention as hereinafter contrasted.

In U.S. Pat. No. 5,634,885, titled Tongue Depressor with Lollipop Holder, invented by Amnon Kiro, tongue depressor is described which includes a blade and a holder. One end of the blade functions as a conventional tongue depressor. The other end of the blade accommodates the holder which functions to receive and grasp the stick of a lollipop. In another configuration, the tongue depressor includes a blade for restraining the tongue of the patient, the blade being formed with a slot shaped to accommodate the stick of the lollipop.

In U.S. Pat. No. 5,318,009, titled Illuminated Tongue Depressor, invented by Herbert L. Robinson, an illuminated tongue depressor is described which includes a handle having a battery-operated light source and a depressor blade selectively coupled with or uncoupled from the handle for discard and replacement. The blade includes a length extending longitudinally between a proximal end and a distal end and laterally between opposite sides, and includes upper and lower surfaces, a peripheral edge between the upper and lower surfaces defining a relatively thin blade thickness, a root adjacent the proximal end of the blade, a tip at the distal end of the blade, and a light conducting material between the upper and lower surfaces and extending along the blade from the root too at least a portion of the peripheral edge at the tip of the blade. A light directing configuration formed on the distal end directs the light transmitted by the light conducting material toward the area to be examined and includes an elevated portion at the tip and a support leg on both sides thereof for contacting the patient's tongue during use to maintain the elevated portion above the portion of the tongue immediately in front of the tip. The handle includes a movable arm and associate contact forming a switch which is automatically closed energizing the light source when the blade is moved into a coupled position, and automatically opened de-energizing the light source when the blade is moved to an uncoupled position.

In Patent Number D305,797, titled Tongue Depressor, invented by Herbert L. Robinson, an ornamental design for a tongue depressor, as shown and described.

In U.S. Pat. No. 4,807,599, titled Illuminating Tongue Depressor, invented by Herbert L. Robinson and Allen S. Epstein, an illuminating tongue depressor includes a handle having a battery-operated light source and a depressor blade selectively coupled with or uncoupled from the handle for discard and replacement, the blade being constructed of a light-conducting synthetic resin material of relatively thin cross-section and being arched laterally to resist bending along the length thereof, the blade including a light-receiving surface at the proximal end thereof for juxtaposition with the light source and a light-directing configuration at the distal end thereof for directing light conducted from the light source and projected from the distal end area to be inspected during use of the tongue depressor in the examination of a patent.

In U.S. Pat. No. 4,643,172 Taff, et. al. Feb. 17, 1987 Luminescent tongue depressor Inventors: Barry E. Taff; and Kenneth Stoller, a luminescent tongue depressor is described having a luminiferous depressor element connected to a handle. Means to illuminate the depressor element is preferably a monatomic gas contained within a vacuum tube positioned within the handle element of the device. A concave reflector element directs and concentrates the light toward the depressor element. A rechargeable power source electrically connected to the sealed vacuum tube acts to provide light to the depressor element and its surrounding area. Light dispersing grooves in the depressor element act to disperse the light transmitted from the light source through the depressor element in the mouth. In an alternative embodiment of the device, the depressor element pivots to lie substantially flat against the base of the handle element when not in use.

The above patented inventions differ from the present invention because they fail to describe or claim at least one combination of the following features depicted in the present invention. In the present invention a tongue depressor is coated with a candy flavoring. The coating is lacking in the prior art patents.

Numerous innovations for a candy coated tongue depressor have been provided in the prior art that are adapted to be used. Even though these innovations may be suitable for the specific individual purposes to which they address, they would not be suitable for the purposes of the present invention as heretofore described.

### SUMMARY OF INVENTION

The present invention is a tongue depressor which has a flavored coating. The coating is palatable to young children. The present invention functions to obtain an increase in the cooperation of small children while being examined by medical personnel.

The types of problems encountered in the prior art are candy is molded into the tongue depressor which adds a manufacturing step and increases the cost. Further, in the prior art there is a risk of the candy coming loose from the tongue depressor creating a choking risk.

In the prior art, unsuccessful attempts to solve this problem were attempted namely; a tongue depressor adapted to receive a lollipop handle or a candy element molded into the surface. However, the problem was solved by the present invention because the tongue depressor is coated with a flavoring after manufacture. Since the coating is thin the choking risk due to the candy member coming lose is eliminated.

The present invention went contrary to the teaching of the art by coating with a flavoring rather than adding a candy element.

The present invention solved a long felt need for a flavored tongue depressor which is inexpensive to produce and assists in getting a child's cooperation during an examination.

Accordingly, it is an object of the present invention to provide a tongue depressor which aids in the examination of a patient's mouth and throat.

More particularly, it is an object of the present invention to provide a tongue depressor having a flavoring.

In keeping with these objects, and with others which will become apparent hereinafter, one feature of the present invention resides, briefly stated, in tongue depressor front and a tongue depressor rear.

When the candy coated tongue depressor is designed in accordance with the present invention, children are more cooperative during an examination.

In accordance with another feature of the present invention, a flavored coating is applied to one end of the tongue depressor.

Another feature of the present invention is that a candy coating is selected which has solidifying properties at or below 40 degrees Celsius.

Yet another feature of the present invention is that the tongue depressor front is dipped into candy which is elevated in temperature above 40 degrees Celsius.

Still, another feature of the present invention is that cooling of the candy coated tongue depressor bonds the candy coating to the surface.

The novel features which are considered characteristic for the invention are set forth in the appended claims. The invention itself, however, both as to its construction and its method of operation, together with additional objects and advantages thereof, will be best understood from the following description of the specific embodiments when read and understood in connection with the accompanying drawings.

### BRIEF DESCRIPTION OF THE DRAWINGS

FIG. 1 is a top view of a candy coated tongue depressor.

FIG. 2 is a cross-sectional view of a candy coated tongue depressor along line 2-2.

FIG. 3 is a diagrammatic representation of a method of manufacturing a candy coated tongue depressor.

### DETAILED DESCRIPTION OF THE PREFERRED EMBODIMENT

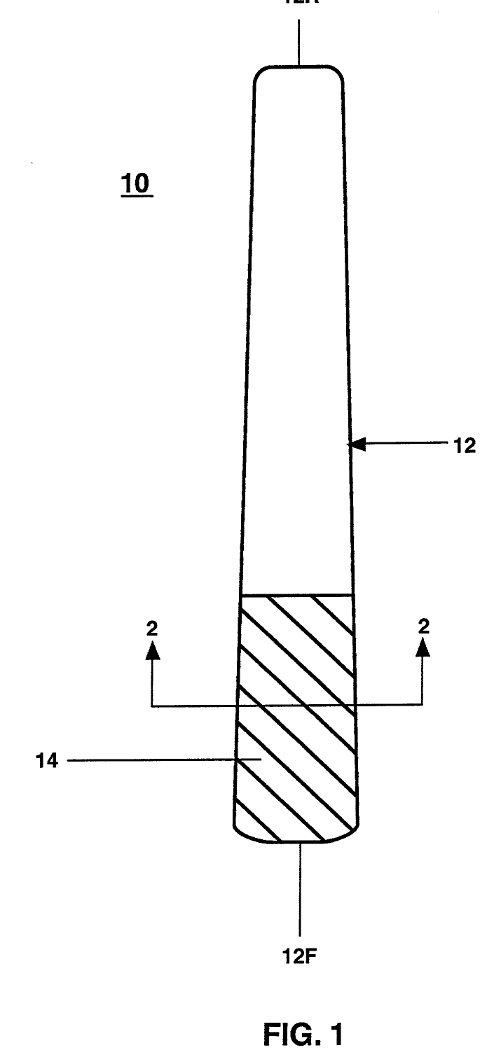

Firstly, referring to FIG. 1 which is a top view of a candy coated tongue depressor 10. The candy coated tongue depressor 10 comprises a tongue depressor 12 which compromises a holeless outer surface, a tongue depressor front 12F and a tongue depressor rear 12R. The tongue depressor 12 is manufactured from an edible material. The edible material is selected from a group consisting of candy, chocolate, fruit, fruit composite, vegetable, vegetable composite, flour, flour composite, ice, flavored ice, meat, meat composite, poultry, poultry composite, fish, and fish composite.

The tongue depressor 12, as an alternative, is manufactured from a non-edible material. The non-edible material is selected from a group consisting of wood, wood composite, plastic, plastic composite, rubber, rubber composite, metal, and metal alloy.

The candy coated tongue depressor 10 further comprises a candy 14 coating securely affixed to the outer surface of the tongue depressor rear 12R. The candy 14 is hard candy or chocolate.

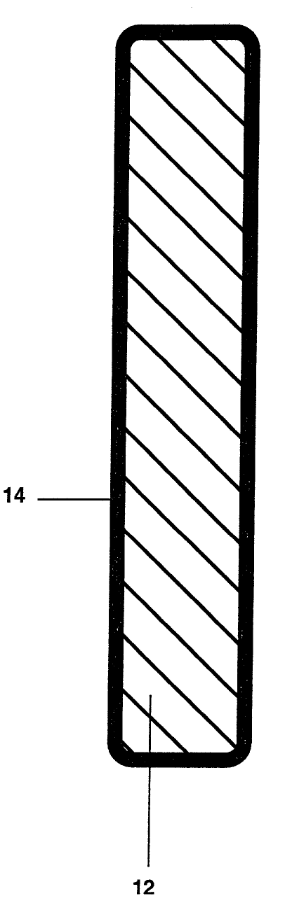

Secondly, referring to FIG. 2 which is a cross-sectional view of a candy coated tongue depressor 10 along line 2-2. A tongue depressor 12 comprises an outer surface. A candy 14 is hard candy or chocolate. When the tongue depressor 12 is dipped into molten candy 14, removed and allowed to cool, the candy 14 is securely bonded to the tongue depressor 12 on an outer surface.

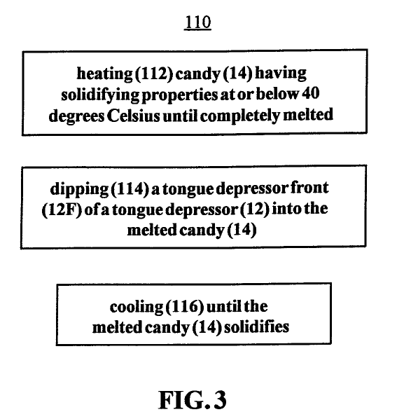

Finally, referring to FIG. 3 which is diagrammatic representation of a method 110 of manufacturing a candy coated tongue depressor 10. Firstly before beginning the manufacturing process the tongue depressor 12 is selected from an edible material or non-edible material. The edible material is selected from a group consisting of candy, chocolate, fruit, fruit composite, vegetable, vegetable composite, flour, flour composite, ice, flavored ice, meat, meat composite, poultry, poultry composite, fish, and fish composite. The non-edible material is selected a group consisting of wood, wood composite, plastic, plastic composite, rubber, rubber composite, metal, and metal alloy. Secondly, the candy 14 is selected from hard candy or chocolate.

A method 110 of manufacturing a candy coated tongue depressor 10 consists of the following steps:

(a) heating 112 candy 14 having solidifying properties at or below 40 degrees Celsius until completely melted;

(b) dipping 114 a tongue depressor front 12F of a tongue depressor 12 is manufactured from an edible material into the melted candy 14;

(c) cooling 116 until the melted candy 14 solidifies which securely attaches the candy 14 to the tongue depressor front 12F.

It will be understood that each of the elements described above, or two or more together, may also find a useful application in other types of constructions differing from the type described above.

While the invention has been illustrated and described as embodied in a candy coated tongue depressor, it is not intended to be limited to the details shown, since it will be understood that various omissions, modifications, substitutions and changes in the forms and details of the device illustrated and in its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

What is claimed:

1. A candy coated tongue depressor manufactured from an edible material comprising:
(a) a tongue depressor which comprises a holeless outer surface, a tongue depressor front and a tongue depressor rear; and,
(b) a candy coating securely affixed to the outer surface of the tongue depressor front.

2. The candy coated tongue depressor as described in claim 1, wherein the edible material is selected from a group consisting of candy, chocolate, fruit, fruit composite, vegetable, vegetable composite, flour, flour composite, ice, flavored ice, meat, meat composite, poultry, poultry composite, fish, and fish composite.

3. A method of manufacturing a candy coated tongue depressor manufactured from an edible material consisting of the following steps:
(a) heating candy having solidifying properties at or below 40 degrees celsius until completely melted;
(b) dipping a holeless tongue depressor front of a tongue depressor into the melted candy; and
(c) cooling until the melted candy solidifies.

4. The Method of manufacturing a candy coated tongue depressor as described in claim 3, wherein the edible material is selected from a group consisting of candy, chocolate, fruit, fruit composite, vegetable, vegetable composite, flour, flour composite, ice, flavored ice, meat, meat composite, poultry, poultry composite, fish, and fish composite.
